(19) United States
(12) Patent Application Publication
(10) Pub. No.: US 2025/0127661 A1
(43) Pub. Date: Apr. 24, 2025

(54) Therapeutic Recovery Device For Medical Rehabilitation

(71) Applicant: Franklin Robert Hill, Jacksonville, FL (US)

(72) Inventor: Franklin Robert Hill, Jacksonville, FL (US)

(21) Appl. No.: 19/006,346

(22) Filed: Dec. 31, 2024

Related U.S. Application Data
(63) Continuation-in-part of application No. 17/892,023, filed on Aug. 19, 2022, now Pat. No. 12,213,863.

(51) Int. Cl. A61F 13/05

(52) U.S. Cl. CPC A61F 13/05

(57) ABSTRACT

The present disclosure provides generally for a therapeutic recovery device for medical rehabilitation and travel. The therapeutic recovery device may comprise a compression device. The compression device may provide compressive force that provides relief and comfort during travel. The therapeutic recovery device may comprise at least one securing mechanism. The therapeutic recovery device may comprise one or more straps. The one or more straps may allow the therapeutic recovery device to be secured in a plurality of orientations. The therapeutic recovery device may comprise at least one holder. When the therapeutic recovery device comprises at least one holder, the holder may comprise one or more pockets.

### CROSS-REFERENCE TO RELATED APPLICATIONS

This application is a continuation in part of currently pending U.S. Nonprovisional patent application Ser. No. 17/892,023, filed Aug. 19, 2022, and titled “THERAPEUTIC RECOVERY DEVICE FOR MEDICAL REHABILITATION”, the entire contents of which are incorporated in this application by reference.

### BACKGROUND

Every year, hundreds of thousands of sternotomies are performed in the United States. A sternotomy is an incision used in cardiothoracic surgery to provide access directly to the thoracic cavity through the sternum. In other words, a sternotomy is a cut down the middle of the patient's chest to perform surgeries on the heart, lungs, and other areas in the chest. Sternotomies are most commonly used in open heart surgeries, and their usage in cardiothoracic surgeries is not likely to decrease, as cardiovascular and lung diseases continue to increase year by year. The other region of the torso, the abdomen, also sees its fair share of invasive surgeries such as appendectomies, colectomies, and other intestinal procedures. The recovery process following any invasive procedure done on the torso can be long and uncomfortable.

The recovery process following an open-heart surgery typically requires a patient to cough often and engage in deep breathing exercises. Because a sternotomy requires the sternum to be broken, an injury that takes several weeks to heal, actions such as deep breathing, coughing, and sneezing can prove to be quite painful for the patient. In addition to physical discomfort and pain, everyday actions can also cause complications in the healing process. To close a sternotomy, the sternum is wired shut, but with too much uncontrolled coughing and sneezing, the wires may loosen and compromise the healing process.

Medical professionals can prescribe medication to ease a patient's pain and discomfort while they recover from a sternotomy, but there is not much else they can do for the patient's physical ailments until they are able to begin their rehabilitation. To provide comfort to recovering sternotomy patients, they are often given rehabilitative pillows to reduce pain caused by coughing, breathing, and sneezing.

The act of embracing a rehabilitative pillow while coughing, breathing, or sneezing can dampen the forces associated with these events and reduce the amount of pain and discomfort felt by the patient. Aside from aiding the patient's comfort, the dampening effect created by the rehabilitative pillows is used as a way to counter the pressure caused by coughing and sneezing that could potentially cause damage to the surgical site.

A sternotomy takes a physical toll on a patient and requires an extended stay in a hospital. After a surgery, patients often find themselves fatigued and physically limited in their mobility, strength, and dexterity while they recover. Because of the physical toll that is left by the procedure; patients often find themselves too fatigued to constantly hold a rehabilitative pillow against their chest or to continue to be reaching for it every time they need to cough or sneeze.

When a rehabilitative pillow gets dropped or otherwise moved to an out-of-reach location, the patient must then exert themselves rather abruptly to suppress their coughs or sneezes. Although patients benefit from the physical and emotional effects of the rehabilitative pillow, by having to exert extra effort for every cough or sneeze, the effects may prove to be more detrimental than beneficial.

### SUMMARY OF THE DISCLOSURE

What is needed is a therapeutic recovery device that can provide post-surgical relief without requiring high levels of exertion from a patient. A therapeutic recovery device that can supply consistent compression without requiring the patient to exert consistent levels of pressure on the therapeutic recovery device is also desired.

In some aspects, a therapeutic recovery device may comprise a compression device, wherein the compression device is configured to be pressed against at least a portion of a body of a user to exert a compressive force against the at least a portion of the body of the user, the compression device comprising: an exterior surface, wherein the exterior surface faces away from the body of the user during use; an interior surface, wherein the interior surface is on the opposite surface of the compression device from the exterior surface and faces towards the body of the user during use; a first distal end, wherein a first edge of the exterior surface and a first edge of the interior surface converge at the first distal end; a second distal end, wherein a second edge of the exterior surface and a second edge of the interior surface converge at the second distal end and the second distal end is opposite to the first distal end; two straight side portions on opposing sides of the compression device, the two straight side portions extending from the first distal end to the second distal end; and a first holder, wherein the first holder comprises a first retaining portion and is attached to the interior surface at three or more attachment locations, wherein the first holder is configured to receive at least one of: at least one limb of the user, at least one appendage of the user, or at least a portion of a luggage handle, wherein the first holder comprises a first opening configured between adjacent attachment locations; a second holder, wherein the second holder comprises a second retaining portion and is attached to the interior surface perpendicular to the first holder at three or more attachment locations, wherein the second holder is configured to receive at least one of: at least one limb of the user, at least one appendage of the user, or at least a portion of a luggage handle, wherein the second holder comprises at least a second opening configured between adjacent attachment locations.

In some aspects wherein at least a portion of the therapeutic recovery device, such as the compression device, may comprise at least one sensor, the at least one sensor may provide feedback on a user's health. The therapeutic recovery device may comprise one or more straps. The one or more straps may allow the therapeutic recovery device to be secured in one of a plurality of potential orientations.

The therapeutic recovery device may comprise at least one holder. When the therapeutic recovery device comprises a holder, the holder, or any other portion of the therapeutic recovery device, may comprise one or more pockets. The therapeutic recovery device may interface with at least one external device. The therapeutic recovery device may comprise a cover. The therapeutic recovery device may comprise at least one internal material. When the therapeutic recovery device comprises a cover, the therapeutic recovery device may comprise at least one internal material that may be removably insertable

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1A illustrates a front view of an exemplary therapeutic recovery device, according to some embodiments of the present disclosure.

FIG. 1B illustrates a rear view of an exemplary therapeutic recovery device, according to some embodiments of the present disclosure.

FIG. 1C illustrates a rear view of an exemplary therapeutic recovery device, according to some embodiments of the present disclosure.

FIG. 2A illustrates a front view of an exemplary therapeutic recovery device comprising straps, according to some embodiments of the present disclosure.

FIG. 2B illustrates a rear view of an exemplary therapeutic recovery device comprising straps, according to some embodiments of the present disclosure.

FIG. 3A illustrates a front view of an exemplary therapeutic recovery device comprising an insertable material, according to some embodiments of the present disclosure.

FIG. 3B illustrates a front view of an exemplary therapeutic recovery device comprising an insertable material, according to some embodiments of the present disclosure.

FIG. 4A illustrates a front view of an exemplary therapeutic recovery device interfacing with an external device, according to some embodiments of the present disclosure.

FIG. 4B illustrates a front view of an exemplary therapeutic recovery device interfacing with an external device, according to some embodiments of the present disclosure.

FIG. 5A illustrates a front view of an exemplary therapeutic recovery device interfacing with luggage, according to some embodiments of the present disclosure.

FIG. 5B illustrates a side view of an exemplary therapeutic recovery device interfacing with luggage, according to some embodiments of the present disclosure.

FIG. 6A illustrates a side view of an exemplary therapeutic recovery device interfacing with luggage, according to some embodiments of the present disclosure.

FIG. 6B illustrates a side view of an exemplary therapeutic recovery device interfacing with luggage, according to some embodiments of the present disclosure.

The Figures are not necessarily drawn to scale, as their dimensions can be varied considerably without departing from the scope of the present disclosure.

### DETAILED DESCRIPTION

In the following sections, detailed descriptions of examples and methods of the disclosure will be given. The description of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Holder: As used herein, refers to an amount of material attached to an interior side of a compression device of a therapeutic recovery device with at least one opening for the purpose of at least partially receiving and resting therein an arm, hand, or any other limb or appendage of a user, or one or more portions thereof, in the holder while retaining compressive force against the body of the user via the compression device. In some embodiments, the holder may comprise both horizontally-oriented and vertically-oriented openings to provide utility regardless of the orientation of the therapeutic recovery device.

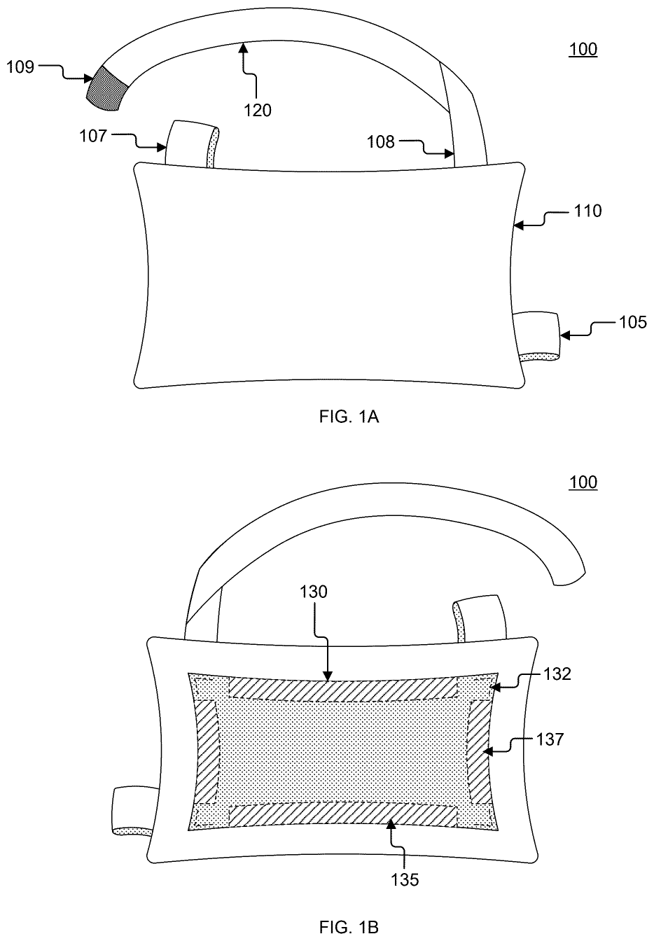

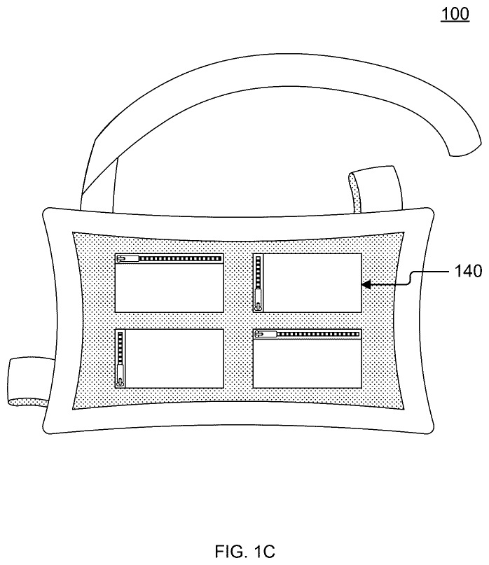

Referring now to FIGS. 1A-C, front and rear views of an exemplary therapeutic recovery device 100 are illustrated. In some embodiments, the therapeutic recovery device 100 may comprise one or more straps 120. In some implementations, the therapeutic recovery device 100 may comprise a plurality of straps 120. In some aspects, the therapeutic recovery device 100 may comprise a compression device 110 configured to be pressed against at least a portion of the body of a user to exert a compressive force against the at least a portion of the body of the user, such as, by way of example and not limitation, the user's torso.

In some embodiments, the therapeutic recovery device 100 may comprise at least one holder 130. In some implementations, the therapeutic recovery device 100 may comprise at least one pocket 140. In some aspects, the therapeutic recovery device 100 may comprise a plurality of pockets 140.

In some embodiments, one or more straps 120 may allow the compression device 110 to be secured in one of a plurality of potential positions or orientations. For example, an extended strap 120 may protrude from one corner of the compression device 110 with the option to interface with either of two separate securing mechanisms 105 on both adjacent corners of the compression device 110.

By connecting to the first securing mechanism 105, the compression device 110 may hang from the strap 120 in a vertical orientation. By detaching from the first securing mechanism 105 and attaching to the second securing mechanism 105, the compression device 110 may hang from the strap 120 in a horizontal orientation.

In some implementations, at least one of the straps 120 may extend around and embrace at least a portion of a user's body, such as the torso, to maintain the compression device 110 in a desired location or position. In some aspects, the holder 130 may assist in securing the therapeutic recovery device 100 in place in addition to or instead of strap(s) 120.

In some aspects, the holder 130 may provide a location to place at least a portion of a limb or appendage, such as a user's arm and/or hand, to keep the compression device 110 close to the body of the user. The increased proximity of the compression device 110 may assist in providing compression from the compression device 110 that reduces pain and discomfort that may be experienced after surgery. In some embodiments, the holder 130 may allow the user to passively retain the compression device 110 against the user's body while allowing one hand to remain free for purposes other than engaging the compression device 110.

In some implementations, the holder 130 may comprise at least one opening. In some embodiments, the holder 130 may comprise at least one vertically-oriented opening and at least one horizontally-oriented opening. In some aspects, a plurality of vertical and horizontal openings may allow the therapeutic recovery device 100 to be retained in any one of a plurality of potential orientations.

For example, the vertical openings may allow the holder 130 to secure an arm while the compression device 110 is in a vertical orientation. If the compression device 110 is switched to a horizontal orientation, then the horizontal openings may allow the holder 130 to secure an arm while the compression device 110 is in the horizontal orientation.

In some embodiments, the holder 130 may comprise a method of securing or closing, or both, any unused openings. As an illustrative example, the holder 130 may comprise at least one fastening element, such as, by way of example and not limitation, a hook-and-loop fastener, snap-fit element, zipper, clamp, clasp, clip, snap, pin, hole, or any similar mechanism, as well as any combination thereof, to close or secure any unused openings.

In some implementations, the therapeutic recovery device 100 may comprise one or more pockets 140. In some embodiments, the pockets 140 may be attached to or integrated with the compression device 110, such as, for example and not limitation, by being attached to an exterior surface of the compression device 110.

In some implementations, the pockets 140 may be connected to or integrated with the holder 130. In some aspects, the pockets 140 may comprise at least one fastening element. In some embodiments, a plurality of pockets 140 may comprise a combination of vertical and horizontal orientations.

The combination of vertical and horizontal orientations may provide accessible pockets 140 to a user regardless of the orientation of the compression device 110. In some implementations, one or more fastening elements may retain items within pockets 140 that are oriented in a position perpendicular to their constructed orientation upon the compression device 110.

For example, a vertically-oriented pocket 140 upon the compression device 110 may, because of a fastening element in the form of a zipper, retain items within the pocket 140 even when the compression device 110 is placed in a horizontal orientation.

In some aspects, the compression device 110 may comprise an exterior surface, wherein the exterior surface faces away from the body of the user during use. In some implementations, the compression device 110 may comprise an interior surface, wherein the interior surface is on the opposite surface of the compression device 110 from the exterior surface and faces towards the body of the user during use.

In some embodiments, the compression device 110 may comprise a first distal end where a first edge of the exterior surface and a first edge of the interior surface converge. In some implementations, the compression device 110 may comprise a second distal end where a second edge of the exterior surface and a second edge of the interior surface converge, and wherein the second distal end is opposite to the first distal end.

In some aspects, the compression device 110 or one or more other portions of the therapeutic recovery device 100 may comprise at least one internal material, such as, by way of example and not limitation, cotton, foam, polyester, or any similar at least partially pliable materials, as well as any combination thereof.

In some embodiments, the at least one internal material may at least partially comprise a weighted material or one or more weighted elements. In some embodiments, the at least one internal material may be removably insertable into at least a portion of the therapeutic recovery device 100, such as, by way of example and not limitation, the compression device 110.

In some aspects, the compression device 110 may comprise two substantially straight side portions on opposing sides of the compression device 110, the two substantially straight side portions extending from the first distal end to the second distal end.

In some embodiments, the compression device 110 may comprise at least one holder 130, wherein the at least one holder 130 comprises a retaining portion and is attached to or integrated with the interior surface of the compression device, wherein the at least one holder 130 is configured to at least partially receive at least a portion of at least one of a user's limbs and/or appendages, such as at least a portion of one of the user's hands and/or arms.

In some implementations, each of the one or more straps 120 may comprise a first end and a second end. In some embodiments, the first end and/or the second end of each strap 120 may be removably attachable to the compression device 110 via at least one securing mechanism 105. By way of example and not limitation, the securing mechanism 105 may comprise a loop of material or fabric, a clamp, a clasp, a clip, at least one hook-and-loop fastener, a hook, a hole, a buckle, or any similar element or mechanism, as well as any combination thereof.

In some aspects, one or both of the first end and the second end of each strap 120 and/or one or more securing mechanisms 105 may remain securely attached to the compression device 110 in a non-removable fashion, such as, by way of example and not limitation, by being sown to, stitched to, adhered to (such as via one or more adhesives), or integrated with the compression device 110.

The first and/or second end of each strap 120 or securing mechanism 105 may extend from or be securely attached to any appropriate portion of the therapeutic recovery device 100, including but not limited to the first distal end, the second distal end, one or both of the substantially straight side portions, or at least one corner of the compression device 110.

In some implementations, the at least one securing mechanism 105 may be configured to adjust a length of a usable portion of each strap 120, thereby adjusting how tightly the compression device 110 is pressed against the body of a user and simultaneously adjusting the amount of compressive force the compression device 110 exerts upon the user's body.

For example, in aspects wherein the securing mechanism 105 may comprise a buckle, the buckle may be alterable between an open state and a secured state, wherein a portion of at least one strap 120 may be slidable through the buckle when the buckle is in the open state, thereby allowing the length of the usable portion of the strap 120 to be adjusted. By way of example and not limitation, a usable portion of a strap 120 may comprise a portion of the strap 120 that is available to encircle, enclose, or embrace at least a portion of a user's body.

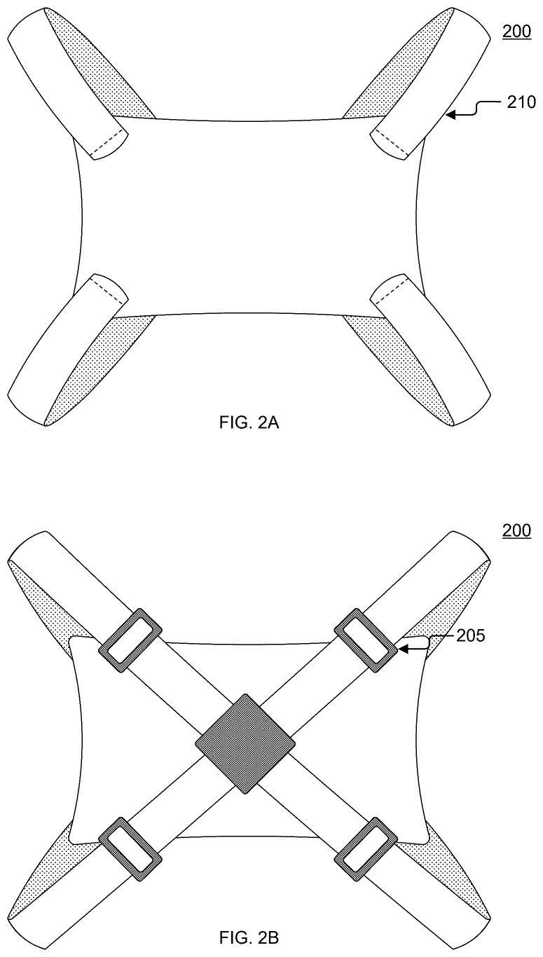

Referring now to FIGS. 2A-B, front and rear views of an exemplary therapeutic recovery device 200 comprising straps 220 are illustrated. In some embodiments, the therapeutic recovery device 200 may comprise a plurality of straps 220. In some implementations, the therapeutic recovery device 200 may comprise a compression device 210.

In some aspects, the therapeutic recovery device 200 may comprise at least one securing mechanism 205. In some embodiments, at least one of the plurality of straps 220 may be adjustable. In some implementations, at least one of the plurality of straps 220 may be adjustable via the securing mechanism 205.

As an illustrative example, at least one of the straps 220 may comprise a tightening buckle that allows a user to tighten one or more loops of strap 220 fixed to the compression device 210 to increase the proximity of the compression device 210 to the user when worn. Securing the therapeutic recovery device 200 via adjustable straps 220 may allow the therapeutic recovery device 200 to be held in a compressive state against the body of the user without the use of the user's hands. This may allow the user to use their hands for other purposes rather than be occupied by engaging the compression device 210.

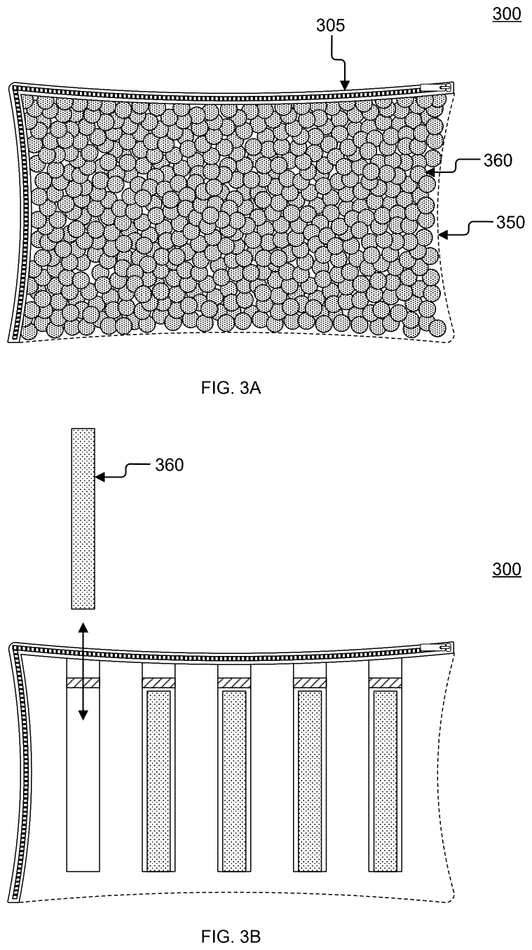

Referring now to FIGS. 3A-B, front views of an exemplary therapeutic recovery device 300 comprising an insertable material 360 are illustrated. In some embodiments, the therapeutic recovery device 300 may comprise a compression device 310. In some implementations, the therapeutic recovery device 300 may comprise a cover 350. In some aspects, the therapeutic recovery device 300 may comprise at least one internal removably insertable material 360. In some embodiments, the therapeutic recovery device 300 may comprise at least one securing mechanism 305.

In some implementations, the cover 350 may comprise the insertable material 360. In some aspects, the cover 350 may be removable. In some embodiments, the cover 350 may attach to the therapeutic recovery device 300 via at least one securing mechanism 305, such as a zipper, as a non-limiting example.

In some implementations, the insertable material 360 may be inserted directly into at least one portion of the compression device 310. In some aspects, the insertable material 360 may be inserted into preconfigured slots within the compression device 310 that are created to receive the insertable material 360.

In some embodiments, the insertable material 360 or other internal material may improve the comfort provided by the therapeutic recovery device 300. For example, an insertable material 360 that at least partially comprises a weighted material or one or more weighted elements may provide comfort similar to a weighted blanket.

As another example, the insertable material 360 may comprise a material or object that may retain a fixed temperature or that may change temperature, such as, by way of example and not limitation, one or more heating elements (such as, for example, one or more hot or heat packs at least partially comprising an amount of magnesium sulfate or calcium chloride), an amount of cooling fabric or cooling gel (such as, for example, sodium polyacrylate), as well as any similar elements or any combination thereof.

Controlling the temperature of the therapeutic recovery device 300 may make wearing the therapeutic recovery device 300 more comfortable or may aid with recovery by applying temperatures conducive for reducing swelling, as non-limiting examples.

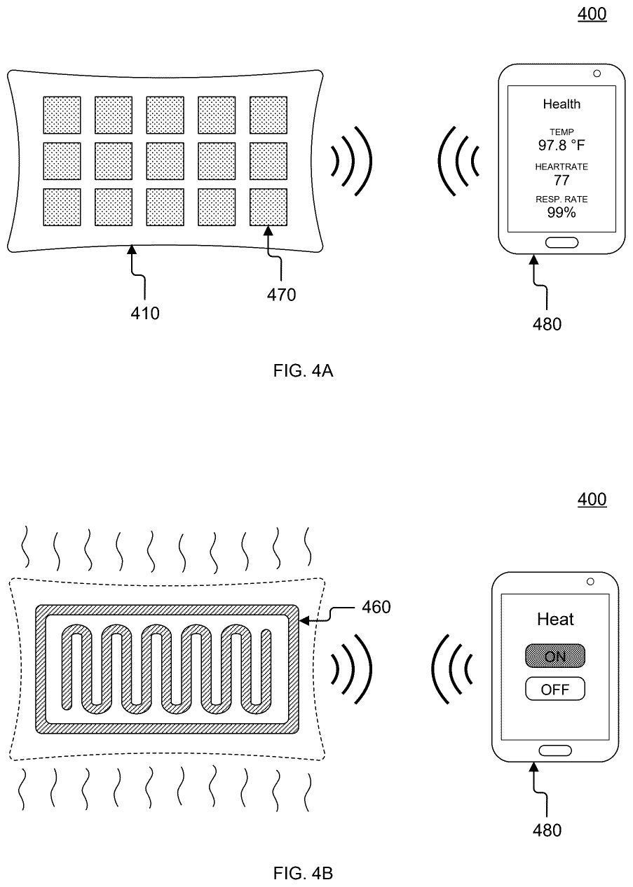

Referring now to FIGS. 4A-B, front views of an exemplary therapeutic recovery device 400 interfacing with an external device 480 are illustrated. In some embodiments, at least a portion of the therapeutic recovery device 400, such as, by way of example and not limitation, a compression device 410, may comprise at least one sensor 470. In some aspects, the at least one sensor 470 may be attached to or integrated with an outer portion or cover of the therapeutic recovery device 400.

In some implementations, the therapeutic recovery device 400 may comprise a plurality of sensors 470. In some embodiments, the therapeutic recovery device 400 may be configured to interface with at least one external device 480. In some implementations, the therapeutic recovery device 400 may comprise at least one internal removably insertable material 460. In some aspects, the at least one sensor 470 may comprise at least one of: a pulse oximeter, a thermometer, a heart rate monitor, an accelerometer, a pressure sensor, and a motion sensor, as well as any similar device.

In some embodiments, at least one of the sensors 470 may be configured to provide or transmit biofeedback data or other information associated with a user to the external device 480, such as oxygen (O2) levels, body temperature, heart rate, and restlessness during sleeping, as a list of non-limiting examples.

For example, at least one of the sensors 470 may measure a heart rate when the

compression device 410 is in close contact with the user. As another example, the sensors 470 may comprise at least one sensor 470 that detects oxygen levels of the user while the therapeutic recovery device 400 is close to the user while sleeping. By way of example and not limitation, the user's oxygen levels may be detected by at least one sensor 470 in the form of a pulse oximeter or any similar oxygen detection or measurement device.

In some implementations, the external device 480 may be configured to communicate with the therapeutic recovery device 400 in a manner sufficient to alter or adjust a state (such as, for example and not limitation, a temperature) of the insertable material 460 or other internal material.

As an example, the insertable material 460 may comprise one or more heating or cooling elements, such as, by way of example and not limitation, one or more heating coils, that may be activated and controlled via a software application on a cellular phone, smartphone, tablet, or similar portable or desktop computing device communicatively coupled to a controller connected to or associated with the insertable material 460 or other internal material.

In some embodiments, the controller and/or one or more wires or other circuitry elements that may be associated therewith may be at least partially configured within a housing. In some aspects, the therapeutic recovery device 400 may comprise at least one power supply, such as, by way of example and not limitation, at least one battery; at least one solar cell or other photovoltaic element; at least one cord, cable, or wire configured to receive an amount of alternating current or direct current electricity, or any similar power supply or source, as well as any combination thereof.

In some implementations, the at least one power supply and any circuitry elements that may be associated therewith may be at least partially configured within the controller housing or within a separate independent housing. The at least one power supply may be configured to power one or more electrical or electromechanical components of the therapeutic recovery device 400, including but not limited to any sensors 470, heating or cooling elements, controllers, or circuitry elements that may be configured or associated with the therapeutic recovery device 400.

In some aspects, one or more sensors 470 may be configured to prompt activation of the internal insertable material 460 or other internal material. In such aspects, the relevant sensor(s) 470 may be communicatively coupled, either directly or indirectly, either wired or wirelessly, to the controller associated with the insertable material 460.

In some embodiments, activation of the insertable material 460 or other internal material may be at least partially based on a predetermined threshold value set by and received from the external device 480.

Continuing the previous example, the therapeutic recovery device 400 may comprise at least one sensor 470 in the form of a thermometer or other temperature sensor that, based on a predetermined temperature set within the application on the computing device, may be configured to activate the heating coils of the internal insertable material 460 to adjust the temperature of the therapeutic recovery device 400 when the temperature sensor detects that the temperature of the therapeutic recovery device 400 has decreased below the predetermined temperature.

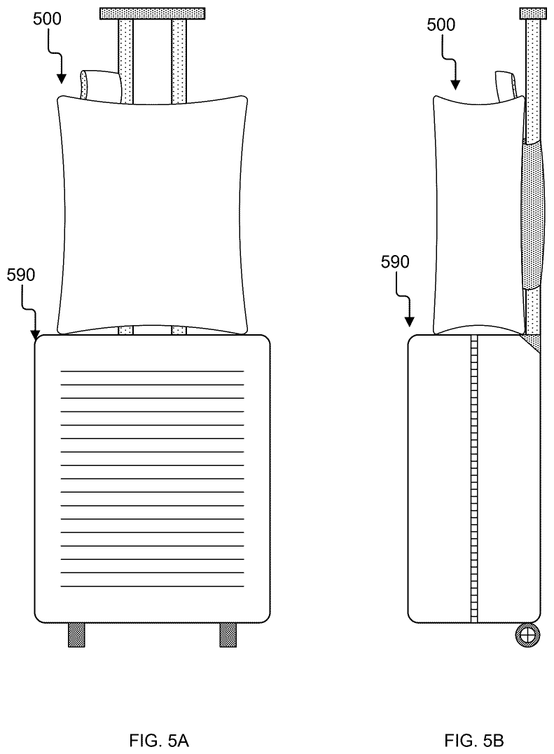

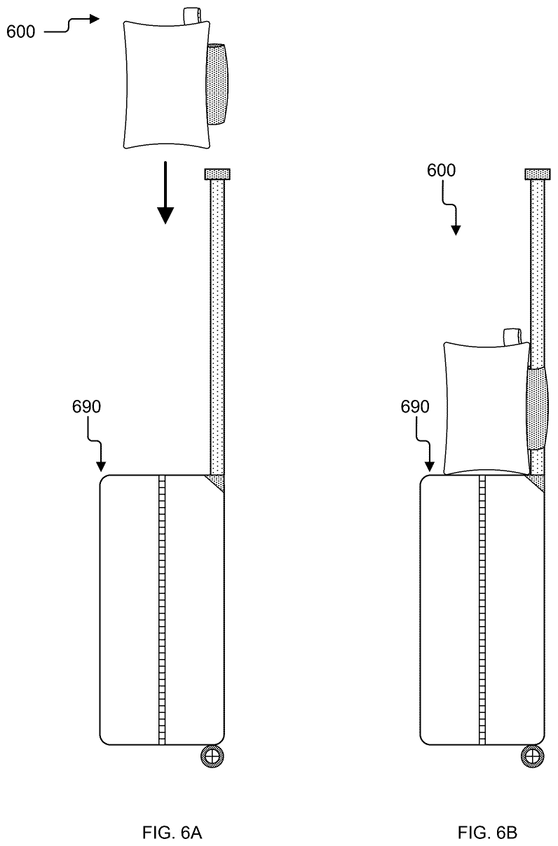

Referring now to FIGS. 5A-5B, an exemplary therapeutic recovery device 500 interfacing with luggage 590, according to some embodiments of the present disclosure, is illustrated. Referring now to FIGS. 6A-6B, an exemplary therapeutic recovery device 600 interfacing with luggage 690, according to some embodiments of the present disclosure, is illustrated. In some aspects, a therapeutic recovery device 500, 600 may fit over at least a portion of a handle of luggage 590, 690 in at least two orientations. A first holder may allow the therapeutic recovery device 500 to fit over the handle of luggage 590 in a vertical orientation. A second holder may allow the therapeutic recovery device 600 to fit over the handle of luggage 690 in a horizontal orientation, perpendicular to the vertical orientation.

The options in orientation may allow a user to travel with the most flexibility. For example, a user may be utilizing a tote in addition to their luggage 690 and may need room on the handle for both the tote and the therapeutic recovery device 600. Traveling with the therapeutic recovery device 500, 600 may give the user the portability of recovery, which may allow a user to travel with limited pain. For example, traveling on an airplane or train may be bumpy, and a user may benefit from a therapeutic recovery device 500, 600 to limit pain caused by the ride.

### Conclusion

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described components and systems can generally be integrated together in a single product or packaged into multiple products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

Reference in this specification to “one embodiment,” “an embodiment,” any other phrase mentioning the word “embodiment”, “aspect”, or “implementation” means that a particular feature, structure, or characteristic described in connection with the embodiment is included in at least one embodiment of the disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional.

Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

What is claimed is:

1. A therapeutic recovery device comprising:
a compression device, wherein the compression device is configured to be pressed against at least a portion of a body of a user to exert a compressive force against the at least a portion of the body of the user, the compression device comprising:
an exterior surface, wherein the exterior surface faces away from the body of the user during use;
an interior surface, wherein the interior surface is on the opposite surface of the compression device from the exterior surface and faces towards the body of the user during use;
a first distal end, wherein a first edge of the exterior surface and a first edge of the interior surface converge at the first distal end;
a second distal end, wherein a second edge of the exterior surface and a second edge of the interior surface converge at the second distal end and the second distal end is opposite to the first distal end;
two straight side portions on opposing sides of the compression device, the two straight side portions extending from the first distal end to the second distal end; and
a first holder, wherein the first holder comprises a first retaining portion and is attached to the interior surface at three or more attachment locations, wherein the first holder is configured to receive at least one of: at least one limb of the user, at least one appendage of the user, or at least a portion of a luggage handle, wherein the first holder comprises a first opening configured between adjacent attachment locations;
a second holder, wherein the second holder comprises a second retaining portion and is attached to the interior surface perpendicular to the first holder at three or more attachment locations, wherein the second holder is configured to receive at least one of: at least one limb of the user, at least one appendage of the user, or at least a portion of a luggage handle, wherein the second holder comprises at least a second opening configured between adjacent attachment locations.

2. The therapeutic recovery device of claim 1, wherein the therapeutic recovery device further comprises one or more straps, wherein each of the one or more straps comprises a first end and a second end.

3. The therapeutic recovery device of claim 2, wherein the therapeutic recovery device further comprises at least one securing mechanism, wherein the at least one securing mechanism extends from at least one of: the first distal end, the second distal end, at least one corner of the compression device, and at least one of the two substantially straight side portions.

4. The therapeutic recovery device of claim 3, wherein at least one of: the first end and the second end of at least one of the one or more straps is removably attachable to the compression device via the at least one securing mechanism.

5. The therapeutic recovery device of claim 2, wherein the first end and the second end of the one or more straps is securely attached to at least one of: the first distal end, the second distal end, at least one corner of the compression device, and at least one of the two substantially straight side portions.

6. The therapeutic recovery device of claim 1, wherein the compression device further comprises one or more pockets, wherein the one or more pockets are attached to the exterior surface.

7. The therapeutic recovery device of claim 1, wherein the at least one securing mechanism is configured to adjust a length of a usable portion of at least one of the one or more straps.

8. The therapeutic recovery device of claim 1, wherein the compression device further comprises at least one internal material.

9. The therapeutic recovery device of claim 1, wherein the therapeutic recovery device further comprises a cover.

10. The therapeutic recovery device of claim 9, wherein the at least one internal material is removably insertable.

11. The therapeutic recovery device of claim 8, wherein the at least one internal material at least partially comprises a weighted material.

12. The therapeutic recovery device of claim 1, wherein the compression device comprises at least one sensor.

13. The therapeutic recovery device of claim 12, wherein the therapeutic recovery device is configured to interface with at least one external device.

14. The therapeutic recovery device of claim 13, wherein the at least one sensor is configured to transmit data to the at least one external device.

15. The therapeutic recovery device of claim 14, wherein the therapeutic recovery device further comprises at least one internal material, wherein the at least one sensor is configured to activate the at least one internal material based on a predetermined value received from the at least one external device.

16. The therapeutic recovery device of claim 8, wherein the at least one internal material is configured to adjust a temperature of the therapeutic recovery device.

17. The therapeutic recovery device of claim 12, wherein the at least one sensor comprises at least one of: a pulse oximeter, a thermometer, a heart rate monitor, an accelerometer, pressure sensor, and a motion sensor.

18. The therapeutic recovery device of claim 16, wherein the at least one internal material comprises at least one of: one or more heating elements, an amount of cooling fabric, and an amount of cooling gel.

19. The therapeutic recovery device of claim 1, wherein the at least one holder comprises at least one opening.

20. The therapeutic recovery device of claim 1, wherein the at least one holder comprises at least one vertically-oriented opening and at least one horizontally-oriented opening.
